(19) United States
(12) Patent Application Publication
(10) Pub. No.: US 2023/0167489 A1
(43) Pub. Date: Jun. 1, 2023

(54) FLOW CELLS AND METHODS OF USE THEREOF

(71) Applicant: 10x Genomics, Inc., Pleasanton, CA (US)

(72) Inventors: Yiran ZHANG, Castro Valley, CA (US); Siyuan XING, Newark, CA (US); Rajiv BHARADWAJ, Pleasanton, CA (US)

(21) Appl. No.: 18/070,887

(22) Filed: Nov. 29, 2022

Related U.S. Application Data
(60) Provisional application No. 63/283,641, filed on Nov. 29, 2021.

(51) Int. Cl. C12Q 1/6841; C12Q 1/6837

(52) U.S. Cl. CPC C12Q 1/6841; C12Q 1/6837

(57) ABSTRACT

The present invention features devices and methods for supplying a biological sample with a fluid. In various embodiments, a device includes a first layer and a second layer. The first layer includes a window having a thickness of about 1 μm to about 1000 μm. The first and second layers reversibly seal to form a flow path having an inlet and an outlet and bounded in part by the window.

### FIELD OF THE INVENTION

The present disclosure generally relates to sample devices for imaging of samples (e.g., biological samples). In particular, the present disclosure relates to sample devices configured to form a closed flow cell around the sample after the device is assembled and allow for imaging in an analytical system, e.g., an instrument having integrated optics and fluidics modules (e.g., an in situ analysis system).

### BACKGROUND

Devices and methods for sample analysis, in situ sequencing, and in situ hybridization typically involve providing reagents to a sample in a well and imaging the sample. Providing precise reagents and controlling sample quality can be challenging. Therefore, improved methods and devices for sample analysis are needed.

### SUMMARY OF THE INVENTION

In one aspect, the invention features a device that includes a first layer and a second layer. The first layer includes a window having a thickness of about 1 μm to about 1000 μm, and the first and second layers reversibly seal to form a flow path having an inlet and an outlet and bounded in part by the window.

In some embodiments, a thickness of the first layer bounding the window is greater than the thickness of the window.

In some embodiments, the thickness of the first layer bounding the window is from about 1 μm to about 1 mm. In some embodiments, the thickness of the first layer bounding the window is from about 1 μm to about 500 μm, from about 1 μm to about 250 μm, from about 10 μm to about 100 μm, from about 100 μm to about 500 μm, from about 100 μm to about 300 μm, from about 100 μm to about 200 μm, from about 120 μm to about 190 μm, or from about 150 μm to about 180 μm. In some embodiments, the thickness of the first layer bounding the window is from about 1 μm to about 10 μm, e.g., about 1 μm, 2 μm, 3 μm, 4 μm, 5 μm, 6 μm, 7 μm, 8 μm, 9 μm, or 10 μm, e.g., from about 10 μm to about 100 μm, e.g., about 10 μm, 20 μm, 30 μm, 40 μm, 50 μm, 60 μm, 70 μm, 80 μm, 90 μm, or 100 μm, e.g., from about 100 μm to about 1 mm, e.g., about 100 μm, 200 μm, 300 μm, 400 μm, 500 μm, 600 μm, 700 μm, 800 μm, 900 μm, or 1 mm. In some embodiments, the thickness of the first layer bounding the window is about 0.17 mm.

In some embodiments, the thickness of the window is from about 1 μm to about 1 mm. In some embodiments, the thickness of the window is from about 1 μm to about 500 μm, from about 1 μm to about 250 μm, from about 10 μm to about 100 μm, from about 100 μm to about 500 μm, from about 100 μm to about 300 μm, from about 100 μm to about 200 μm, from about 120 μm to about 190 μm, or from about 150 μm to about 180 μm. In some embodiments, the thickness of the window is from about 1 μm to about 10 μm, e.g., about 1 μm, 2 μm, 3 μm, 4 μm, 5 μm, 6 μm, 7 μm, 8 μm, 9 μm, or 10 μm, e.g., from about 10 μm to about 100 μm, e.g., about 10 μm, 20 μm, 30 μm, 40 μm, 50 μm, 60 μm, 70 μm, 80 μm, 90 μm, or 100 μm, e.g., from about 100 μm to about 1 mm, e.g., about 100 μm, 200 μm, 300 μm, 400 μm, 500 μm, 600 μm, 700 μm, 800 μm, 900 μm, or 1 mm. In some embodiments, the thickness of the window is about 0.17 mm.

In some embodiments, the flow path includes a void in a surface of the first or second layer.

In some embodiments, the first layer and the second layer are configured to seal via an adhesive, conformal contact, or capillary force.

In some embodiments, the first layer includes a plurality of windows having a thickness of about 1 μm to about 1000 μm that are physically separated. In some embodiments, the thickness of the windows is from about 1 μm to about 500 μm, from about 1 μm to about 250 μm, from about 10 μm to about 100 μm, from about 100 μm to about 500 μm, from about 100 μm to about 300 μm, from about 100 μm to about 200 μm, from about 120 μm to about 190 μm, or from about 150 μm to about 180 μm. In some embodiments, the thickness of the windows is from about 1 μm to about 10 μm, e.g., about 1 μm, 2 μm, 3 μm, 4 μm, 5 μm, 6 μm, 7 μm, 8 μm, 9 μm, or 10 μm, e.g., from about 10 μm to about 100 μm, e.g., about 10 μm, 20 μm, 30 μm, 40 μm, 50 μm, 60 μm, 70 μm, 80 μm, 90 μm, or 100 μm, e.g., from about 100 μm to about 1 mm, e.g., about 100 μm, 200 μm, 300 μm, 400 μm, 500 μm, 600 μm, 700 μm, 800 μm, 900 μm, or 1 mm.

In some embodiments, each of the plurality of windows is separated by a region of the first layer having a greater thickness than each window.

In some embodiments, each of the plurality of windows is separated by a region of the first layer including a hydrophobic pattern.

In some embodiments, the flow path is bounded in part by a hydrophobic pattern on the first and/or second layer.

In some embodiments, the second layer includes a hydrophobic surface texture or surface pattern opposite the window.

In some embodiments, the first and/or second layer includes an elastomer.

In some embodiments, the second layer includes the inlet and/or the outlet.

In another aspect, the invention features a system that includes a device as described herein and a clamp configured to apply a force on the device to maintain the seal.

In another aspect, the invention features a method for assembling a device by providing a device or system as described herein; applying a sample to the window; and reversibly contacting the first layer and the second layer and forming a fluid tight seal and the flow path through which fluid can flow.

In some embodiments, the contacting step includes applying a force on the device with the clamp.

In another aspect, the invention features a method for detection with a device or system as described herein. The method includes providing the device or system, applying a sample to the window, and flowing a fluid through the flow path. The fluid may include an oligonucleotide probe that hybridizes to a template nucleic acid in the sample, and the method may further include detecting the oligonucleotide probe.

In some embodiments, the oligonucleotide probe comprises an optical label.

In some embodiments, the optical label is a fluorescent label.

In some embodiments, the method further includes flowing a fluid comprising a replicating enzyme and a plurality of nucleotide triphosphates (NTPs) or oligonucleotides to the sample.

In some embodiments, the replicating enzyme replicates the template nucleic acid with the NTPs.

### BRIEF DESCRIPTION OF THE DRAWINGS

FIG. 1 is a schematic drawing illustrating a cross-sectional side view of a sample device, according to various embodiments.

FIG. 2 is a schematic drawing illustrating a cross-sectional side view of a sample device, according to various embodiments.

FIG. 3 is a schematic drawing illustrating a cross-sectional side view of a sample device having multiple chambers, according to various embodiments.

FIGS. 4A and 4B are schematic drawings illustrating top views of sample devices having multiple windows, according to various embodiments.

FIG. 5 is a schematic drawing illustrating a cross-sectional side view of sample device, according to various embodiments.

FIG. 6A is a schematic drawing illustrating a cross-sectional side view of a sample device having a hydrophobic region and a reagent region, according to various embodiments.

FIG. 6B is a schematic drawing illustrating a surface having a hydrophobic region and a reagent region, according to various embodiments.

### DETAILED DESCRIPTION OF THE INVENTION

The invention provides methods, devices, and systems for manipulating, viewing, and imaging a biological sample, e.g., supplied with fluids, e.g., to perform a variety of analyses. Biological samples are typically visualized and manipulated on a glass slide (e.g., a cover slip), which is thin and fragile. The devices and systems described herein include a first layer having a thickness and with a window for viewing a sample in the device. The device includes a window (e.g., similar to a cover slip thickness) for optimal optical parameters (e.g., when using a microscope), while implementing other thicker components (e.g., the sides of the device) to provide a durable and less fragile device that can be easily assembled by a user prior to sample manipulation. Furthermore, the device can be optimized with a fluidic channel shape built into the geometry of the window for optimized flow path conditions. The devices provide enhanced mechanical stability, flatness, ease of assembly and alignment, robustness to handling without breaking, and improved ease of disassembling and reusing parts. Finally, the devices and systems can still provide a fluid tight seal that does not require a gasket.

### Exemplary Devices

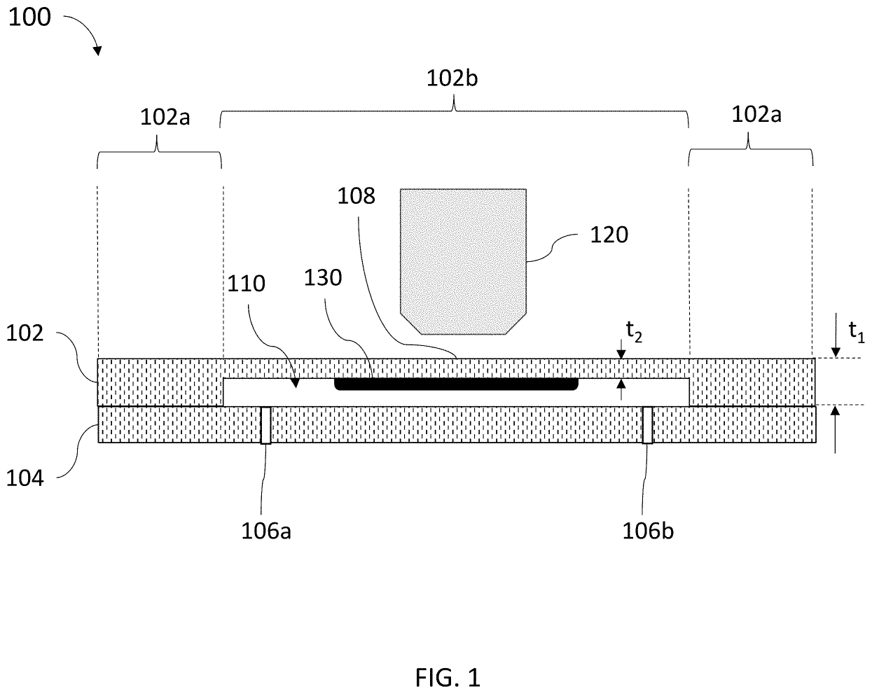

FIG. 1 illustrates a cross-sectional side view of a sample device 100. As shown in FIG. 1, the sample device 100 includes a first layer 102 having a biological sample 130 (e.g., cells, tissues, a matrix, etc.) disposed thereon. The first layer 102 is disposed on a second layer 104 such that a chamber 110 is formed therebetween. In various embodiments, the first layer 102 includes a window 108 configured to allow imaging of the biological sample 130 via an objective lens 120 of a microscope (e.g., in an opto-fluidic instrument). In various embodiments, the chamber 110 is substantially sealed from leaks, e.g., is watertight and/or airtight. In various embodiments, the first layer 102 is affixed to the second layer 104 via, for example, an adhesive, a fixation device (e.g., magnets, screws, clips, latches, connectors, etc.), capillary action of a fluid between the two contacting surfaces, gravity, etc. In various embodiments, the second layer 104 includes at least one inlet 106a and at least one outlet 106b. In various embodiments, one or more reagents flow through the inlet 106a to thereby immerse the biological sample 130 and, subsequently or simultaneously, out of the outlet 106b to thereby remove spent reagent. In various embodiments, the first layer 102 includes one or more first portions 102a having a thickness t₁. In various embodiments, the first portion 102a extends around the perimeter of and thereby defines the wall of the chamber 110. In various embodiments, the first layer 102 includes a second portion 102b having a thickness t₂ that is less than the thickness t₁. In various embodiments, the inlet 106a and/or the outlet 106b includes a valve configured to control flow of a fluid (e.g., reagent) in and/or out of the chamber 110.

In various embodiments, the chamber 110 is dimensioned to have a maximum volume of about 100 μl to about 5 mL (e.g., about 100 μl to 1 mL, e.g., about 100 μl, 200 μl, 300 μ, l400 μl, 500 μl, 600 μl, 700 μl, 800 μl, 900 μl, or 1 ml, e.g., about 1 mL to about 5 mL, e.g., about 1 mL, 1.25 mL, 1.5 mL, 1.75 mL, 2 mL, 2.25 mL, 2.5 mL, 2.75 mL, 3 mL, 3.25 mL, 3.5 mL, 3.75 mL, 4 mL, 4.25 mL, 4.5 mL, 4.75 mL, or 5 mL).

In various embodiments, the chamber 110 is dimensioned to have a maximum volume of about 500 μl to about 2 mL. In various embodiments, the chamber 110 is dimensioned to have a maximum volume of about 750 μl to about 1.5 mL. In various embodiments, the chamber 110 is dimensioned to have a maximum volume of about 1 mL to about 1.25 mL.

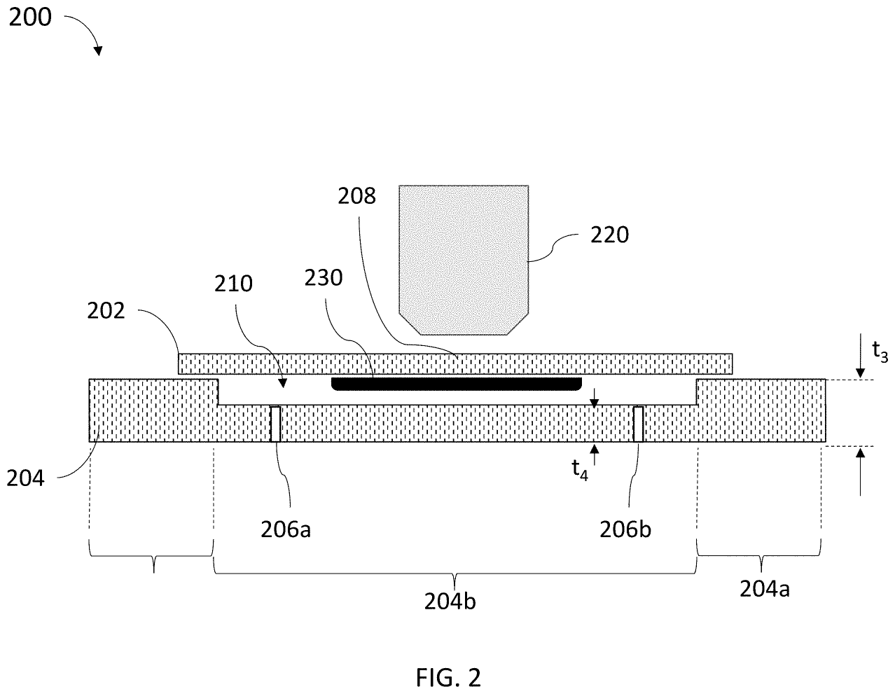

FIG. 2 illustrates a cross-sectional side view of a sample device 200. Similar to the sample device 100 of FIG. 1, the sample device 200 of FIG. 2 includes a first layer 202 having a biological sample 230 (e.g., cells, tissues, a matrix, etc.) disposed thereon. The first layer 202 is disposed on a second layer 204 such that a chamber 210 is formed therebetween. In various embodiments, the first layer 202 includes a window 208 configured to allow imaging of the biological sample 230 via an objective lens 220 of a microscope (e.g., in an opto-fluidic instrument). In various embodiments, the chamber 210 is substantially sealed from leaks, e.g., is watertight and/or airtight. In various embodiments, the first layer 202 is affixed to the second layer 204 via, for example, an adhesive, a fixation device (e.g., magnets, screws, clips, latches, connectors, etc.), capillary action of a fluid between the two contacting surfaces, gravity, etc. In various embodiments, the second layer 204 includes at least one inlet 206a and at least one outlet 206b. In various embodiments, one or more reagents flow through the inlet 206a to thereby immerse the biological sample 230 and, subsequently or simultaneously, out of the outlet 206b to thereby remove spent reagent. In various embodiments, the second layer 204 includes one or more first portion 204a having a thickness t₃. In various embodiments, the first portion 204a extends around the perimeter of and thereby defines the wall of the chamber 210. In various embodiments, the second 204 layer includes a second portion 204b having a thickness t₄ that is less than the thickness t₃. In various embodiments, the inlet 206a and/or the outlet 206b includes a valve configured to control flow of a fluid (e.g., reagent) in and/or out of the chamber 210.

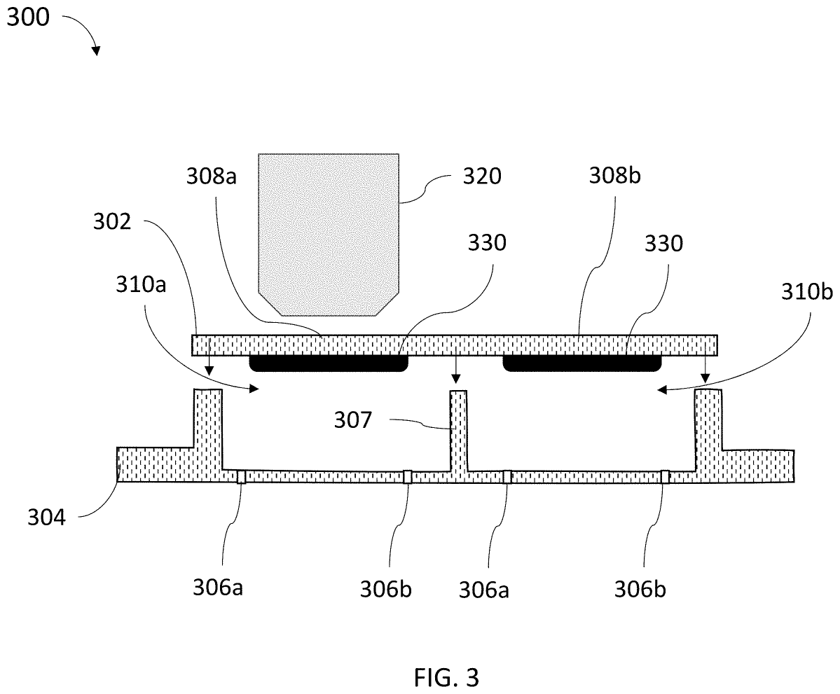

FIG. 3 illustrates a cross-sectional side view of a sample device 300 having multiple chambers 310a, 310b. Similar to the sample devices 100 and 200 of FIGS. 1 and 2, the sample device 300 of FIG. 3 includes a first layer 302 having biological samples 330 (e.g., cells, tissues, a matrix, etc.) disposed thereon. The first layer 302 is disposed on a second layer 304 such that two or more chambers 310a, 310b are formed therebetween. In various embodiments, the first layer 302 includes windows 308a, 308b configured to allow imaging of the biological samples 330 via an objective lens 320 of a microscope (e.g., in an opto-fluidic instrument). In various embodiments, each chamber 310a, 310b is substantially sealed from leaks, e.g., is watertight and/or airtight. In various embodiments, the first layer 302 is affixed to the second layer 304 via, for example, an adhesive, a fixation device (e.g., magnets, screws, clips, latches, connectors, etc.), capillary action of a fluid between the two contacting surfaces, gravity, etc. In various embodiments, the second layer 304 includes at least one divider 307 configured to separate each chamber 310a, 310b and seal against the first layer 302. In various embodiments, the second layer 304 includes at least one inlet 306a and at least one outlet 306b. In various embodiments, each chamber 310a, 310b includes at least one inlet 306a and at least one outlet 306b. In various embodiments, one or more reagents flow through the inlets 306a to thereby immerse the biological samples 330 and, subsequently or simultaneously, out of the outlets 306b to thereby remove spent reagent. In various embodiments, the inlets 306a and/or the outlets 306b include a valve configured to control flow of a fluid (e.g., reagent) in and/or out of the chambers 310a, 310b.

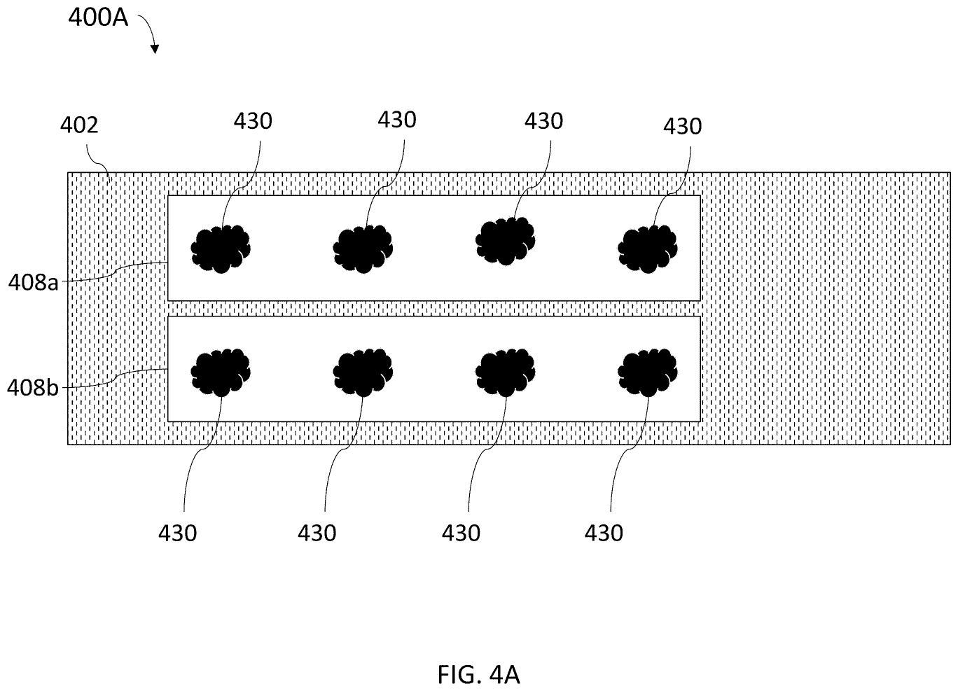

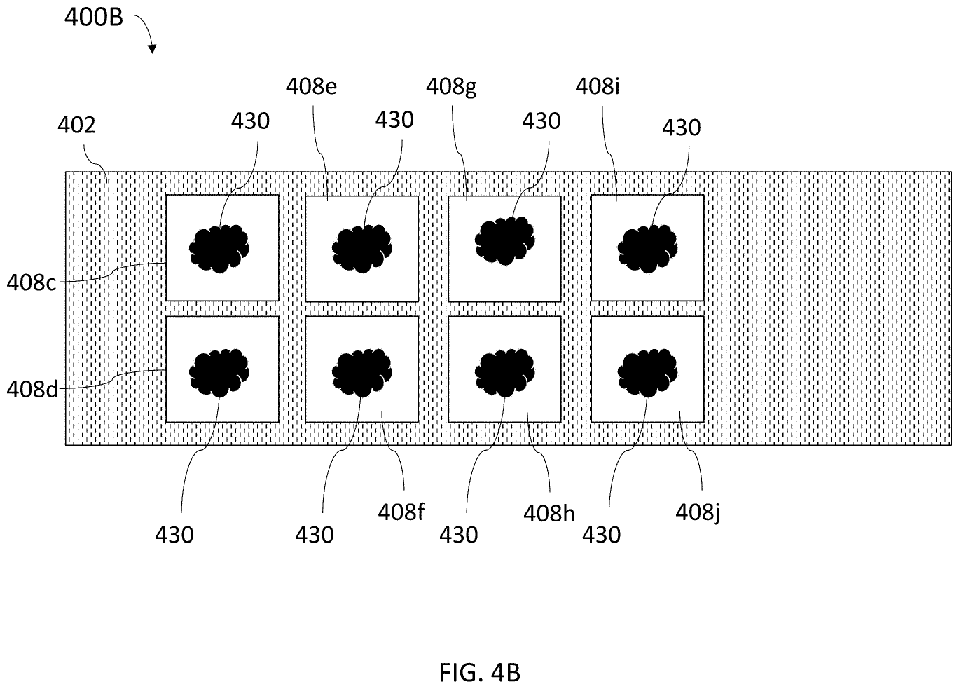

FIGS. 4A and 4B illustrate top views of sample devices 400A, 400B having multiple windows. In some embodiments, the first layer 402 includes a plurality of windows 408a-408b that are physically separated (FIG. 4A) and configured for imaging one or more biological samples 430. As shown in FIG. 4A, sample device 400A has windows 408a, 408b. In some embodiments, each window 408a, 408b may have a long side and a short side, forming a rectangular shape. In some embodiments, one or more biological samples 430 (e.g., two, three, four, etc.) are positioned (e.g., affixed) on each window 408a, 408b of the first layer 402. The plurality of windows 408a-408b may be physically separated, e.g., via a protrusion, a groove, a divider, or other feature present in the device (e.g., in the first layer). In some embodiments, each of the plurality of windows is separated by a region of the first layer that has a greater thickness than the window. In some embodiments, each of the plurality of windows is separated by a region of the first layer that has, for example, a hydrophobic pattern. As shown in FIG. 4B, sample device 400B has windows 408c-408j. In some embodiments, as shown in FIG. 4B, each window 408c-408j is dimensioned such that a single biological sample can be positioned (e.g., affixed) to the area of the window. In some embodiments of any sample device described herein, the windows are of any suitable shape, e.g., rectangular-, square-, circular-, oval-, pentagon-, hexagon-shaped.

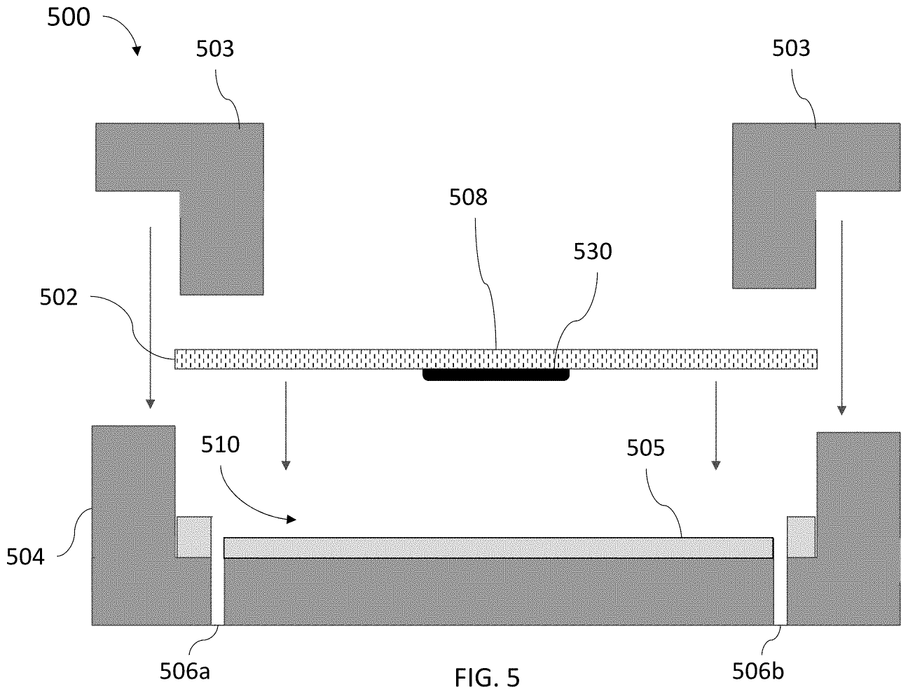

FIG. 5 illustrates a cross-sectional side view of sample device 500. As shown in FIG. 5, the sample device 500 includes a first layer 502 having a biological sample 530 (e.g., cells, tissues, a matrix, etc.) disposed thereon. In various embodiments, the sample device 500 includes a gasket 505. In various embodiments, the gasket 505 is a separate component that is disposed on the second layer 504. In various embodiments, the gasket 505 is an integral part of the second layer 504. In various embodiments, the gasket 505 is made of the same material as the first layer 502 and/or the second layer 504. In various embodiments, the gasket is made of a different material from the first layer 502 and/or the second layer 504. In various embodiments, the gasket 505 is inserted into the second layer 504 during assembly of the sample device 500. In various embodiments, the gasket 505 is formed (e.g., over molded) on the second layer. As shown in FIG. 5, the first layer 502 is disposed on the gasket 505 such that a chamber 510 is formed therebetween. In various embodiments, the first layer 502 includes a window 508 configured to allow imaging of the biological sample 530 via an objective lens of a microscope (e.g., in an opto-fluidic instrument). In various embodiments, the chamber 510 is substantially sealed from leaks, e.g., is watertight and/or airtight. In various embodiments, the first layer 502 is affixed to the second layer 504 via a clamp 503 (and clamp 503 may assist in forming a seal between the first layer 502 and the gasket 505). In various embodiments, the second layer 504 includes at least one inlet 506a and at least one outlet 506b. In various embodiments, one or more reagents flow through the inlet 506a to thereby immerse the biological sample 530 and, subsequently or simultaneously, out of the outlet 506b to thereby remove spent reagent. In various embodiments, the inlet 506a and/or the outlet 506b includes a valve configured to control flow of a fluid (e.g., reagent) in and/or out of the chamber 510.

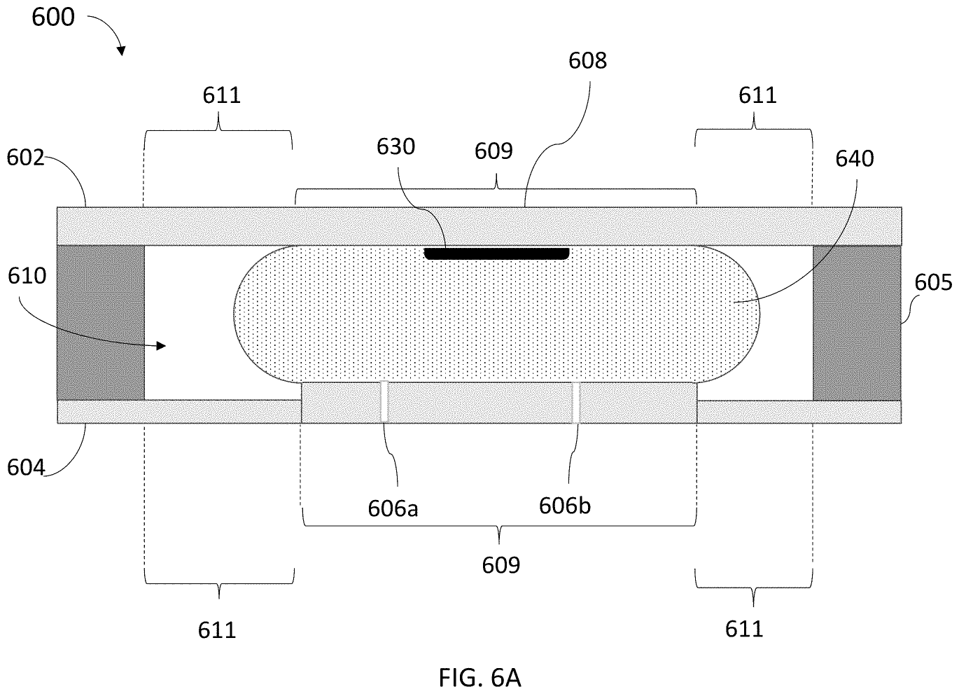

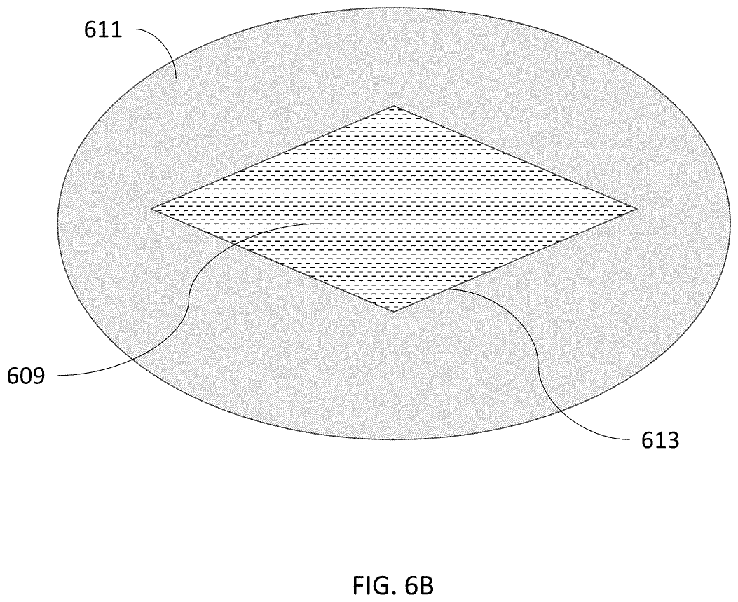

FIG. 6A illustrates a cross-sectional side view of a sample device 600 having a hydrophobic region 611 and a reagent region 609. As shown in FIG. 6A, the sample device 600 includes a first layer 602 having a biological sample 630 (e.g., cells, tissues, a matrix, etc.) disposed thereon. The first layer 602 is disposed on a second layer 604 such that a chamber 610 is formed therebetween. In various embodiments, the sample device includes a gasket 605. In various embodiments, the gasket 605 is integral with the second layer 604. In various embodiments, the first layer 602 includes a window 608 configured to allow imaging of the biological sample 630 via an objective lens of a microscope (e.g., in an opto-fluidic instrument). In various embodiments, the chamber 610 is substantially sealed from leaks, e.g., is watertight and/or airtight. In various embodiments, the first layer 602 is affixed to the second layer 604 via, for example, an adhesive, a fixation device (e.g., magnets, screws, clips, latches, connectors, etc.), capillary action of a fluid between the two contacting surfaces, gravity, etc. In various embodiments, the second layer 604 includes at least one inlet 606a and at least one outlet 606b. In various embodiments, one or more reagents flow through the inlet 606a to thereby immerse the biological sample 630 and, subsequently or simultaneously, out of the outlet 606b to thereby remove spent reagent. In various embodiments, the inlet 606a and/or the outlet 606b includes a valve configured to control flow of a fluid (e.g., reagent) in and/or out of the chamber 610.

As shown in FIG. 6A, the first layer 602 includes a reagent region 609 and a hydrophobic region 611. Similarly, the second layer 604 includes a reagent region 609 and a hydrophobic region 611. In various embodiments, the reagent region 609 has a different thickness than the hydrophobic region 611 for the second layer 604. In various embodiments, the reagent region 609 has a different thickness than the hydrophobic region 611 for the first layer 602 (not shown). In various embodiments, the portion of the second layer 604 that corresponds to the reagent region 609 has a greater thickness than a thickness of the portion of the second layer 604 that corresponds to the hydrophobic region 611. In various embodiments, when reagent 640 is transported into the chamber 610, the reagent 640 contacts only portions of the first layer 602 and the second layer 604 that are the reagent region 609. In various embodiments, the reagent 640 forms a contact angle with the first layer and/or second layer 604 at the interface 613. In various embodiments, the reagent region 609 is dimensioned such that the reagent 640 substantially immerses the biological sample 630.

FIG. 6B illustrates a surface 650 having a hydrophobic region 611 and a reagent region 609. In various embodiments, the reagent region 609 has a hydrophilic surface. For example, the reagent region 609 may have a hydrophilic coating, be chemically treated to have hydrophilic function groups, be made from a hydrophilic material, or be treated to have hydrophilic properties (e.g., via plasma treatment). In various embodiments, the hydrophobic region 611 has a hydrophobic coating (e.g., polytetrafluoroethylene). In various embodiments, the hydrophobic region 611 is made of a material that is substantially hydrophobic (e.g., polytetrafluoroethylene). In various embodiments, the hydrophobic region 611 is treated to have hydrophobic properties. In various embodiments, an interface 613 between the hydrophobic region 611 and the reagent region 609 defines a pinning line where a reagent forms a contact angle with the surface (at least until surface tension is broken). In various embodiments, flow rates of reagent into and out of the chamber are such that the reagent does not spill into the hydrophobic region 611 (e.g., surface tension is not broken). In various, embodiments, the interface 613 is square-, rectangular-, circular-, oval-, pentagon-, or hexagon-shaped, or any other suitable shape to keep reagent within the reagent region 609.

The invention features a device that includes a first layer and a second layer. The first layer includes a window having a thickness of about 1 μm to about 1000 μm, and the first and second layers reversibly seal to form a flow path having an inlet and an outlet and bounded in part by the window (FIG. 1). The thickness of the first layer bounding the window may be greater than the thickness of the window. Alternatively, the thickness of the first layer bounding the window may be substantially the same thickness as the window.

In some embodiments, the thickness of the first layer bounding the window is from about 1 μm to about 1 mm. In some embodiments, the thickness of the first layer bounding the window is from about 1 μm to about 500 μm, from about 1 μm to about 250 μm, from about 10 μm to about 100 μm, from about 100 μm to about 500 μm, from about 100 μm to about 300 μm, from about 100 μm to about 200 μm, from about 120 μm to about 190 μm, or from about 150 μm to about 180 μm. In some embodiments, the thickness of the first layer bounding the window is from about 1 μm to about 10 μm, e.g., about 1 μm, 2 μm, 3 μm, 4 μm, 5 μm, 6 μm, 7 μm, 8 μm, 9 μm, or 10 μm, e.g., from about 10 μm to about 100 μm, e.g., about 10 μm, 20 μm, 30 μm, 40 μm, 50 μm, 60 μm, 70 μm, 80 μm, 90 μm, or 100 μm, e.g., from about 100 μm to about 1 mm, e.g., about 100 μm, 200 μm, 300 μm, 400 μm, 500 μm, 600 μm, 700 μm, 800 μm, 900 μm, or 1 mm. In some embodiments, the thickness of the first layer bounding the window is about 0.17 mm.

The first layer and/or second layer may be any suitable shape, such as a square, rectangle, or circle, so long as to contain and/or visualize the sample. In some embodiments, the length and/or width of the first layer and/or second layer is, independently, from about 1 mm to about 10 cm, e.g., from about 1 mm to about 1 cm, e.g., about 2 mm, 3 mm, 4 mm, 5 mm, 6 mm, 7 mm, 8 mm, 9 mm, or 1 cm, e.g., from about 1 cm to about 10 cm, e.g., about 2 cm, 3 cm, 4 cm, 5 cm, 6 cm, 7 cm, 8 cm, 9 cm, or 10 cm. In some embodiments, the first layer and/or second layer is a coverslip, e.g., having dimensions of about 22 mm by 22 mm (square), about 24 mm by about 50 mm (rectangle), or a circle with diameter of about 12 mm or about 25 mm.

In some embodiments, the thickness of the window is from about 1 μm to about 1 mm. In some embodiments, the thickness of the window is from about 1 μm to about 500 μm, from about 1 μm to about 250 μm, from about 10 μm to about 100 μm, from about 100 μm to about 500 μm, from about 100 μm to about 300 μm, from about 100 μm to about 200 μm, from about 120 μm to about 190 μm, or from about 150 μm to about 180 μm. In some embodiments, the thickness of the window is from about 1 μm to about 10 μm, e.g., about 1 μm, 2 μm, 3 μm, 4 μm, 5 μm, 6 μm, 7 μm, 8 μm, 9 μm, or 10 μm, e.g., from about 10 μm to about 100 μm, e.g., about 10 μm, 20 μm, 30 μm, 40 μm, 50 μm, 60 μm, 70 μm, 80 μm, 90 μm, or 100 μm, e.g., from about 100 μm to about 1 mm, e.g., about 100 μm, 200 μm, 300 μm, 400 μm, 500 μm, 600 μm, 700 μm, 800 μm, 900 μm, or 1 mm. In some embodiments, the thickness of the window is about 0.17 mm.

The window may be any suitable shape, such as a square, rectangle, or circle, in order to visualize the sample. In some embodiments, the length and/or width of the window is, independently, from about 1 mm to about 10 cm, e.g., from about 1 mm to about 1 cm, e.g., about 2 mm, 3 mm, 4 mm, 5 mm, 6 mm, 7 mm, 8 mm, 9 mm, or 1 cm, e.g., from about 1 cm to about 10 cm, e.g., about 2 cm, 3 cm, 4 cm, 5 cm, 6 cm, 7 cm, 8 cm, 9 cm, or 10 cm. In some embodiments, the window is a coverslip, e.g., having dimensions of about 22 mm by 22 mm (square), about 24 mm by about 50 mm (rectangle), or a circle with diameter of about 12 mm or about 25 mm.

When multiple windows are present, each window may have a thickness of about 1 μm to about 1000 μm. In some embodiments, the thickness of each window is from about 1 μm to about 500 μm, from about 1 μm to about 250 μm, from about 10 μm to about 100 μm, from about 100 μm to about 500 μm, from about 100 μm to about 300 μm, from about 100 μm to about 200 μm, from about 120 μm to about 190 μm, or from about 150 μm to about 180 μm. In some embodiments, the thickness of each window is from about 1 μm to about 10 μm, e.g., about 1 μm, 2 μm, 3 μm, 4 μm, 5 μm, 6 μm, 7 μm, 8 μm, 9 μm, or 10 μm, e.g., from about 10 μm to about 100 μm, e.g., about 10 μm, 20 μm, 30 μm, 40 μm, 50 μm, 60 μm, 70 μm, 80 μm, 90 μm, or 100 μm, e.g., from about 100 μm to about 1 mm, e.g., about 100 μm, 200 μm, 300 μm, 400 μm, 500 μm, 600 μm, 700 μm, 800 μm, 900 μm, or 1 mm. In some embodiments, the thickness of each window is about 0.17 mm.

In some embodiments, the first layer and the second layer are configured to form a fluid tight seal, e.g., water- and/or airtight. For example, the layers may be sealed via an adhesive, conformal contact, or by capillary force. In some embodiments, a force is applied to the device to maintain the seal. The device may be configured for use with a clamp to apply a force and maintain the seal (FIG. 5). In some embodiments, the first and/or second layer includes an elastomer, e.g., polydimethylsiloxane (PDMS). The elastomer may assist in allowing the layers to conformally contact each other to maintain the seal.

The device described herein includes a flow path having an inlet and an outlet and bounded in part by the window. The flow path may be any suitable geometry, such as a cylindrical flow path, square flow path, rectangular flow path, or the like. In some embodiments, the flow path includes a void in a surface of the first or second layer. In some embodiments, the flow path is bounded in part by a hydrophobic pattern on the first and/or second layer. The hydrophobic pattern has a boundary that creates a separation between a portion of the first layer and/or second layer containing the flow path and a remaining portion of the first layer and/or second layer that does not include the flow path (FIGS. 6A and 6B). In some embodiments, the second layer includes a hydrophobic surface texture or surface pattern (e.g., pinning lines) opposite the window. In some embodiments, a device with a hydrophobic surface texture or surface pattern does not require a fluid tight seal. As the local surface hydrophobicity is able to restrict the fluid, the device only requires spacers between the layers to maintain a predetermined height of the device (e.g., the flow path), but pressure may not be required to assemble and maintain integrity of the device.

In some embodiments, the width and/or height of the flow path is, independently, from about 1 μm to about 1 cm. In some embodiments, width and/or height of the flow path is, independently, from about 1 μm to about 500 μm, from about 1 μm to about 250 μm, from about 10 μm to about 100 μm, from about 100 μm to about 500 μm, from about 100 μm to about 300 μm, from about 100 μm to about 200 μm, from about 120 μm to about 190 μm, or from about 150 μm to about 180 μm, In some embodiments, the width and/or height of the flow path is, independently, from about 1 μm to about 10 μm, e.g., about 1 μm, 2 μm, 3 μm, 4 μm, 5 μm, 6 μm, 7 μm, 8 μm, 9 μm, or 10 μm, e.g., from about 10 μm to about 100 μm, e.g., about 10 μm, 20 μm, 30 μm, 40 μm, 50 μm, 60 μm, 70 μm, 80 μm, 90 μm, or 100 μm, e.g., from about 100 μm to about 1 mm, e.g., about 100 μm, 200 μm, 300 μm, 400 μm, 500 μm, 600 μm, 700 μm, 800 μm, 900 μm, or 1 mm, e.g., from about 1 mm to about 1 cm, e.g., about 2 mm, 3 mm, 4 mm, 5 mm, 6 mm, 7 mm, 8 mm, 9 mm, or 1 cm.

In some embodiments, the length of the flow path is longer than the width and/or height of the flow path. In some embodiments, the length of the flow path is, e.g., from about 100 μm to about 10 cm, e.g., from about 100 μm to about 1 mm, e.g., about 100 μm, 200 μm, 300 μm, 400 μm, 500 μm, 600 μm, 700 μm, 800 μm, 900 μm, or 1 mm, e.g., from about 1 mm to about 1 cm, e.g., about 1 mm, 2 mm, 3 mm, 4 mm, 5 mm, 6 mm, 7 mm, 8mm, 9 mm, or 1 cm, e.g., from about 1 cm to about 10 cm, e.g., 2 cm, 3 cm, 4 cm, 5 cm, 6 cm, 7 cm, 8 cm, 9 cm, or 10 cm.

The device described herein includes an inlet and an outlet to allow fluid to flow through into and out of the flow path of the device. In some embodiments, the first layer includes the inlet and/or the outlet. In some embodiments, the second layer includes the inlet and/or the outlet. In some embodiments, the first layer includes the inlet, and the second layer includes the outlet. In some embodiments, the second layer includes the inlet, and the first layer includes the outlet.

In some embodiments, the device includes a plurality of inlets and/or outlets. For example, the device may include 1, 2, 3, 4, 5, 6, 7, 8, 9, 10, or more inlets and/or outlets.

In some embodiments, the device includes a plurality of windows. For example, the device may include, 1, 2, 3, 4, 5, 6, 7, 8, 9, 10, 15, 20, 25, 30, 35, 40, 45, 50, or more windows. In some embodiments, each window corresponds to a different flow path. In some embodiments, each window corresponds to a different set of inlets and/or outlets.

In some embodiments, the device includes a plurality of flow paths. For example, the device may include 1, 2, 3, 4, 5, 6, 7, 8, 9, 10, or more flow paths. In such an embodiment, each inlet and/or outlet may correspond to a single flow path. Alternatively, a single inlet and/or outlet may provide access to a plurality of flow paths.

The layers of a device as described herein may be composed of polymeric materials, such as polyethylene or polyethylene derivatives, such as cyclic olefin copolymers (COC), polymethylmethacrylate (PMMA), polydimethylsiloxane (PDMS), polycarbonate, polystyrene, polypropylene, polyvinyl chloride, polytetrafluoroethylene, polyoxymethylene, polyether ether ketone, polycarbonate, polystyrene, or the like. The layers may be fabricated in whole or in part from inorganic materials, such as silicon, or other silica-based materials, e.g., glass, quartz, fused silica, borosilicate glass, metals, ceramics, and combinations thereof. Polymeric device components may be fabricated using any of a number of processes including soft lithography, embossing techniques, micromachining, e.g., laser machining, or in some aspects, injection molding of the layer components that include the defined channels, windows, as well as other structures, e.g., reservoirs, integrated functional components, and the like.

### Surface Properties

The first layer, second layer, and/or the window of the device may have a surface modification, e.g., a surface with a coating, e.g., a hydrophobic coating, or a surface texture. A surface of the device may include a material, coating, or surface texture that determines the physical properties of the device. In particular, the flow of liquids through a device of the invention may be controlled by the device surface properties (e.g., wettability of a liquid-contacting surface). In some cases, a device portion (e.g., a flow cell) may have a surface having a wettability suitable for facilitating liquid flow (e.g., in a channel) or for creating a hydrophobic boundary.

Wetting, which is the ability of a liquid to maintain contact with a solid surface, may be measured as a function of a water contact angle. A water contact angle of a material can be measured by any suitable method known in the art, such as the static sessile drop method, pendant drop method, dynamic sessile drop method, dynamic Wilhelmy method, single-fiber Wilhelmy method, single-fiber meniscus method, and Washburn's equation capillary rise method. The wettability of each surface may be suited to creating a hydrophobic boundary.

For example, portions of the device carrying aqueous phases (e.g., a flow cell) may have a surface material or coating that is hydrophilic or more hydrophilic than an adjacent region, e.g., include a material or coating having a water contact angle of less than or equal to about 90°, and/or the adjacent region may have a surface material or coating that is hydrophobic or more hydrophobic than the flow cell, e.g., include a material or coating having a water contact angle of greater than 70° (e.g., greater than 90°, greater than 95°, greater than 100° (e.g., 95°-120° or 100°-10°). In certain embodiments, the adjacent region may include a material or surface coating that reduces or prevents wetting by aqueous phases.

The device can be designed to have a single type of material or coating throughout. Alternatively, the device may have separate regions having different materials or coatings.

The device surface properties may be those of a native surface (i.e., the surface properties of the bulk material used for the device fabrication) or of a surface treatment. Non-limiting examples of surface treatments include, e.g., surface coatings and surface textures. In one approach, the device surface properties are attributable to one or more surface coatings present in a device portion. Hydrophobic coatings may include fluoropolymers (e.g., AQUAPEL® glass treatment), silanes, siloxanes, silicones, or other coatings known in the art. Other coatings include those vapor deposited from a precursor such as henicosyl-1,1,2,2-tetrahydrododecyldimethyltris(dimethylaminosilane); henicosyl-1,1,2,2-tetrahydrododecyltrichlorosilane (C12); heptadecafluoro-1,1,2,2-tetrahydrodecyltrichlorosilane (C10); nonafluoro-1,1,2,2-tetrahydrohexyltris(dimethylamino)silane; 3,3,3,4,4,5,5,6,6-nonafluorohexyltrichlorosilane; tridecafluoro-1,1,2,2-tetrahydrooctyltrichlorosilane (C8); bis(tridecafluoro-1,1,2,2-tetrahydrooctyl)dimethylsiloxymethylchlorosilane; nonafluorohexyltriethoxysilane (C6); dodecyltrichlorosilane (DTS); dimethyldichlorosilane (DDMS); or 10-undecenyltrichlorosilane (V11); pentafluorophenylpropyltrichlorosilane (C5). Hydrophilic coatings include polymers such as polysaccharides, polyethylene glycol, polyamines, and polycarboxylic acids. Hydrophilic surfaces may also be created by oxygen plasma treatment of certain materials.

A coated surface may be formed by depositing a metal oxide onto a surface of the device. Example metal oxides useful for coating surfaces include, but are not limited to, Al₂O₃, TiO₂, SiO₂, or a combination thereof. Other metal oxides useful for surface modifications are known in the art. The metal oxide can be deposited onto a surface by standard deposition techniques, including, but not limited to, atomic layer deposition (ALD), physical vapor deposition (PVD), e.g., sputtering, chemical vapor deposition (CVD), or laser deposition. Other deposition techniques for coating surfaces, e.g., liquid-based deposition, are known in the art. For example, an atomic layer of Al₂O₃ can be deposited on a surface by contacting it with trimethylaluminum (TMA) and water.

In another approach, the device surface properties may be attributable to surface texture. For example, a surface may have a nanotexture, e.g., have a surface with nanometer surface features, such as cones or columns, that alters the wettability of the surface. Nanotextured surface may be hydrophilic, hydrophobic, or superhydrophobic, e.g., have a water contact angle greater than 150°. Exemplary superhydrophobic materials include Manganese Oxide Polystyrene (MnO₂/PS) nano-composite, Zinc Oxide Polystyrene (ZnO/PS) nano-composite, Precipitated Calcium Carbonate, Carbon nano-tube structures, and a silica nano-coating. Superhydrophobic coatings may also include a low surface energy material (e.g., an inherently hydrophobic material) and a surface roughness (e.g., using laser ablation techniques, plasma etching techniques, or lithographic techniques in which a material is etched through apertures in a patterned mask). Examples of low surface energy materials include fluorocarbon materials, e.g., polytetrafluoroethylene (PTFE), fluorinated ethylene propylene (FEP), ethylene tetrafluoroethylene (ETFE), ethylene chloro-trifluoroethylene (ECTFE), perfluoro-alkoxyalkane (PFA), poly(chloro-trifluoro-ethylene) (CTFE), perfluoro-alkoxyalkane (PFA), and poly(vinylidene fluoride) (PVDF). Other superhydrophobic surfaces are known in the art.

In some cases, the water contact angle of a hydrophilic or more hydrophilic material or coating is less than or equal to about 90°, e.g., less than 80°, 70°, 60°, 50°, 40°, 30°, 20°, or 10°, e.g., 90°, 85°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, or 0°. In some cases, the water contact angle of a hydrophobic or more hydrophobic material or coating is at least 70°, e.g., at least 80°, at least 85°, at least 90°, at least 95°, or at least 100° (e.g., about 100°, 101°, 102°, 103°, 104°, 105°, 106°, 107°, 108°, 109°, 110°, 115°, 120°, 125°, 130°, 135°, 140°, 145°, or about) 150°.

The difference in water contact angles between that of a hydrophilic or more hydrophilic material or coating and a hydrophobic or more hydrophobic material or coating may be 5° to 100°, e.g., 5° to 80°, 5° to 60°, 5° to 50°, 5° to 40°, 5° to 30°, 5° to 20°, 10° to 75°, 15° to 70°, 20° to 65°, 25° to 60°, 30 to 50°, 35° to 45°, e.g., 5°, 6°, 7°, 8°, 9°, 10°, 15°, 20°, 25°, 30°, 35°, 40°, 45°, 50°, 55°, 60, 65°, 70°, 75°, 80°, 85°, 90°, 95°, or 100°.

The above discussion centers on the water contact angle. It will be understood that liquids employed in the devices and methods of the invention may not be water, or even aqueous. Accordingly, the actual contact angle of a liquid on a surface of the device may differ from the water contact angle. Furthermore, the determination of a water contact angle of a material or coating can be made on that material or coating when not incorporated into a device of the invention.

### Medium

Liquid media may be used with a sample as described herein. The liquid media may be aqueous or nonaqueous (e.g., an oil). The liquid medium may contain reagents for preservation of the biological tissue sample. Examples of aqueous liquid media include, e.g., sterile water and phosphate buffered saline. Examples of oils include mineral oil and silicone oils. In embodiments of the invention including reagents in the fluid, a non-aqueous liquid medium may confer the advantage of reducing diffusion of reagents beyond a desired region of the sample.

### Heating and Cooling

Devices of the invention may include a heater and/or cooler, e.g., on or operatively connected to the device, e.g., in the inlet, in thermal contact with a fluid source, or in thermal contact with the sample. Suitable heaters include, but are not limited to, thermoelectric heaters, e.g., thermistors, resistive foil, metal ceramic heaters, thermal tape, etc. Suitable heaters for heating the source of fluid include, but are not limited to, thermoelectric heaters, e.g., thermistors, resistive foil, metal ceramic heaters, thermal tape, a Peltier stage, a TEC controller, etc. Exemplary coolers include high thermal mass or high surface area heat sinks, heat exchangers, Peltier stages, flowing water, a chiller pump, etc.

Heaters and coolers may be configured to supply fluid at appropriate temperatures to perform certain biochemical reactions, e.g., initialization, ligation, DNA melting, annealing, extension, denaturation, etc.

It will be understood that any of the heating sources and temperatures described herein may also be used together. For example, a Peltier stage may be used to heat a source of fluid, while a resistive foil or metal ceramic heater maintains the fluid temperature in the device.

### Reagents

The fluid sources described herein may contain one or more reagents that are delivered to a sample. A fluid source may include one or more (e.g., 2, 3, 4, 5, 6, 7, 8, 9, 10, or more) reagents, or each reagent may be contained in a distinct fluid source. In embodiments in which in situ-based methods are performed, the reagents include, but are not limited to, probes, primers, nucleotide triphosphates (NTPs, e.g., dNTPs), sequencing terminators, dyes, replicating enzymes (e.g., DNA or RNA polymerases, reverse transcriptases, ligases), labels, and the like.

Other reagents that may be provided by a fluid source include, without limitation, a tissue fixing agent, a tissue permeabilizer, such as a solvent (e.g., acetone and methanol) or a detergent (e.g., TRITON X-100, NP-40, TWEEN 20, saponin, digitonin, and Leucoperm).

### Kits and Systems

Devices of the invention may be combined with various external components, e.g., heaters, coolers, detectors, pumps, reservoirs, or controllers, one or more detectors (e.g., one or more lenses (e.g., tube lens), microscope objectives, lasers, spectrometers, etc.), liquid handlers, reagents (e.g., detectable labels, such as nucleic acids, oligonucleotides, ligands, enzymes, proteins, fluorochromes, metal ions, etc., e.g., analyte detection moieties, liquids, particles (e.g., beads), and/or sample) in the form of kits and systems.

In certain embodiments, a kit or system as described herein includes a device as described herein and a clamp configured to apply a force on the device to maintain the seal. The clamp may be used to assemble the device or to maintain the integrity of the device during use. The clamp may be a single integral part or multiple parts. A clamp can include a top plate and a bottom block in which the second layer of the device sits (e.g., before it is sealed with the second layer). The clamp may further include an alignment housing that ensures proper alignment of the layers before sealing (FIG. 5). The clamp, which is used to assemble the device, may be configured to stay attached to the flow cell, e.g., during subsequent use. In such an embodiment, the device may further include a fluidic access port, e.g., to allow access through the clamp to the inlet of the device. Alternatively, the clamp may be removed once the device is assembled.

### Methods

The invention provides methods of supplying a fluid (e.g., including a reagent for sequencing) to a biological sample, such as a tissue. While the fluid is in contact with the tissue, reagents may diffuse from the fluid into the tissue. The methods are advantageous for use in in situ-based methods, such as in situ hybridization and in situ sequencing. Methods of the invention may be particularly advantageous for analysis of a region of interest within a biological tissue sample. The shape and size of the region of interest of the biological tissue may be of any suitable size or shape. For example, a cross sectional dimension of the region of interest may range from 10 μm to 10 mm, e.g., from 1 mm to 5 mm.

A method of the invention for supplying a fluid to a biological tissue sample includes providing a device of the invention. The method further includes placing the biological tissue sample on the window. The method further includes flowing a fluid through the flow path.

In some embodiments, the method further includes providing an oligonucleotide probe in the fluid that hybridizes to a template nucleic acid in the sample. The method may further include detecting the oligonucleotide probe. In some embodiments, the method further includes flowing a fluid (e.g., a second fluid) comprising a replicating enzyme and a plurality of nucleotide triphosphates (NTPs) or oligonucleotides to the sample. The replicating enzyme replicates the template nucleic acid with the NTPs.

In certain embodiments of the method, the fluid flows at a first volumetric flow rate, e.g., to an inlet of the device Suitable volumetric flow rates for the inlet include from about 0.1 nL/s to about 1 μL/s, e.g., about 0.1-1 nL/s (e.g., about 0.1 nL/s, 0.2 nL/s, 0.3 nL/s, 0.4 nL/s, 0.5 nL/s, 0.6 nL/s, 0.7 nL/s, 0.8 nL/s, 0.9 nL/s, or 1 nL/s,), e.g., about 1-10 nL/s (e.g., about 1 nL/s, 2 nL/s, 3 nL/s, 4 nL/s, 5 nL/s, 6 nL/s, 7 nL/s, 8 nL/s, 9 nL/s, or 10 nL/s), e.g., about 10-100 nL/s (e.g., about 10 nL/s, 20 nL/s, 30 nL/s, 40 nL/s, 50 nL/s, 60 nL/s, 70 nL/s, 80 nL/s, 90 nL/s, or 100 nL/s), or, e.g., about 100-1000 nL/s (e.g., about 100 nL/s, 200 nL/s, 300 nL/s, 400 nL/s, 500 nL/s, 600 nL/s, 700 nL/s, 800 nL/s, 900 nL/s, or 1000 nL/s).

In some embodiments, the fluid is aspirated from an outlet of the device. Suitable volumetric flow rates for outlet aspiration include from about 0.1 nL/s to about 1 μL/s, e.g., about 0.1-1 nL/s (e.g., about 0.1 nL/s, 0.2 nL/s, 0.3 nL/s, 0.4 nL/s, 0.5 nL/s, 0.6 nL/s, 0.7 nL/s, 0.8 nL/s, 0.9 nL/s, or 1 nL/s,), e.g., about 1-10 nL/s (e.g., about 1 nL/s, 2 nL/s, 3 nL/s, 4 nL/s, 5 nL/s, 6 nL/s, 7 nL/s, 8 nL/s, 9 nL/s, or 10 nL/s), e.g., about 10-100 nL/s (e.g., about 10 nL/s, 20 nL/s, 30 nL/s, 40 nL/s, 50 nL/s, 60 nL/s, 70 nL/s, 80 nL/s, 90 nL/s, or 100 nL/s), or, e.g., about 100-1000 nL/s (e.g., about 100 nL/s, 200 nL/s, 300 nL/s, 400 nL/s, 500 nL/s, 600 nL/s, 700 nL/s, 800 nL/s, 900 nL/s, or 1000 nL/s).

The ratio of the volumetric flow rate of the inlet, or inlets, to the outlet, or outlets, may be from about 1:1.001 to about 1:10, e.g., about 1:1.001 to about 1:2 (e.g., 1:1.1, 1:1.2, 1:1.3, 1:1.4, 1:1.5, 1:1.6, 1:1.7, 1:1.8, 1:1.9, or 1:2), e.g., about 1:2 to 1:10 (e.g., 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5, or 1:10).

In another embodiment of the method, the first layer and/or second layer seals against each other or via a gasket. When the seal is made, a flow path is formed between the first and second layers. The flow path formed is in fluid communication with a fluid source, e.g., via an inlet to the device. The seal formed may result from, be reinforced by, and/or maintained by mechanical pressure between the first layer and the second layer (e.g., via a clamp, e.g., a magnetic clamp), or by creating negative pressure in the flow path by aspiration through the first outlet.

In some embodiments, the method includes using a plurality of (e.g., 2, 3, 4, 5, 6, 6, 8, 9, 10, or more) fluid sources, each flow source containing a different fluid, e.g., containing a specific reagent, e.g., for in situ-based methods (e.g., in situ hybridization or in situ sequencing), that contact the sample. Each fluid source is in fluid communication with, e.g., fluidically connected to, an inlet of the device. In some embodiments, each fluid source is in fluid communication with, e.g., fluidically connected to, a corresponding inlet of the device. In some embodiments, each fluid source is in fluid communication with, e.g., fluidically connected to, the same inlet, e.g., via a manifold. Alternatively, fluid sources may be sequentially connected and disconnected, either manually or automatically, from being in fluid communication with the same inlet.

Certain methods of the invention include heating or cooling the fluid. Heating or cooling of the fluid can be achieved by heating or cooling the fluid source or the fluid itself, for example by providing a heater or cooler, e.g., thermoelectric device, in the device or fluid source. Heating and or cooling of the fluid may be used to perform temperature-dependent biochemical reactions in the tissue.

Certain methods of the invention include the additional step of obtaining data from a sample, e.g., by optical detection of probes, e.g., using a microscope.

In any embodiment of the invention, the fluid may include a tissue fixing agent, a tissue permeabilizer, an oligonucleotide, NTPs, or a replicating enzyme, e.g., polymerase or ligase.

Also featured is a method for assembling a device by providing a device or system as described herein. The method includes applying a sample to the window and reversibly contacting the first layer and the second layer to form a fluid tight seal and a flow path through which fluid can flow. In some embodiments, the contacting step includes applying a force on the device with a clamp.

### Preparation of Samples

A variety of steps can be performed to prepare a biological tissue sample for analysis. Except where indicated otherwise, the preparative steps described below can generally be combined in any manner to appropriately prepare a particular sample for analysis.

A biological tissue sample can be harvested from a subject (e.g., via surgical biopsy, whole subject sectioning), grown in vitro on a growth substrate or culture dish as a population of cells, or prepared as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., from about 10 μm to about 20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is about 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analyzed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analyzed successively to obtain three-dimensional information about the biological sample.

In some embodiments, the biological tissue sample (e.g., a tissue section as described above) can be prepared by deep freezing at a temperature suitable to maintain or preserve the integrity (e.g., the physical characteristics) of the tissue structure. Such a temperature can be, e.g., less than −20° C., or less than −25° C., −30° C., −40° C., −50° C., −60° C., −70° C., 80° C. −90° C., −100° C., −110° C., −120° C., −130° C., −140° C., −150° C., −160° C., −170° C., −180° C., −190° C., or −200° C. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C. A sample can be snap frozen in isopentane and liquid nitrogen. Frozen samples can be stored in a sealed container prior to embedding.

### Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or padlock probe. In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a padlock probe.

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other hydrogel-formation method known in the art.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

### Staining

To facilitate visualization, biological samples can be stained using a wide variety of stains and staining techniques. In some embodiments, for example, a sample can be stained using any number of stains, including but not limited to, acridine orange (CAS #: 494-38-2), Bismarck brown (e.g., Bismark brown Y, CAS #: 8005-77-4), carmine, Coomassie blue (CAS #: 6104-59-2), cresyl violet (CAS #: 18472-89-4), 4′,6-diamidino-2-phenylindole (DAPI, CAS #: 28718-90-3)), eosin (e.g., eosin Y (CAS #: 17372-87-1) or eosin B (CAS #: 548-24-3)), ethidium bromide (CAS #: 1239-45-8), acid fuchsine (CAS #: 3244-88-0), haematoxylin (CAS #: 517-28-2), Hoechst stains, iodine (e.g., potassium triiodide), methyl green (CAS #: 82-94-0), methylene blue (CAS #: 61-73-4), neutral red (CAS #: 553-24-2), Nile blue (CAS #: 3625-57-8), Nile red (CAS #: 7385-67-3), osmium tetroxide, propidium iodide, rhodamine, or safranin (CAS #: 477-73-6).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Methods of destaining or discoloring a biological sample are known in the art, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### Isometric Expansion

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, Mass.), Label-IT Amine (available from MirusBio, Madison, Wis.) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

### Crosslinking and De-crosslinking

In some embodiments, the biological sample is reversibly cross-linked prior to or during an in situ assay round. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other hydrogel-formation method known in the art. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g., PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, transposase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

### Tissue Permeabilization and Treatment

In some embodiments, a biological sample can be permeabilized to facilitate transfer of analytes out of the sample, and/or to facilitate transfer of species (such as probes) into the sample. If a sample is not permeabilized sufficiently, the amount of analyte captured from the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. Non-chemical permeabilization methods are known in the art. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to opening up DNA in a cell for hybridization by a probe. For example, proteinase K treatment may be used to free up DNA with proteins bound thereto.

### Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g., an affinity binding partner) can be developed. Such a specific binding partner may be a nucleic acid probe (for a nucleic acid analyte) and may lead directly to the generation of a RCA template (e.g., a padlock or other circularizable probe). Alternatively, the specific binding partner may be coupled to a nucleic acid, which may be detected using an RCA strategy, e.g., in an assay which uses or generates a circular nucleic acid molecule which can be the RCA template.

Analytes of particular interest may include nucleic acid molecules, such as DNA (e.g., genomic DNA, mitochondrial DNA, plastid DNA, viral DNA, etc.) and RNA (e.g., mRNA, microRNA, rRNA, snRNA, viral RNA, etc.), and synthetic and/or modified nucleic acid molecules, (e.g., including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g., including but not limited to protein-DNA complexes, which may or may not be covalently bound to one another, and which may be the same or different. Thus, in addition to cells or microorganisms, such a complex analyte may also be a protein complex or protein interaction. Such a complex or interaction may thus be a homo- or hetero-multimer. Aggregates of molecules, e.g., proteins may also be target analytes, for example aggregates of the same protein or different proteins. The analyte may also be a complex between proteins or peptides and nucleic acid molecules such as DNA or RNA, e.g., interactions between proteins and nucleic acids, e.g., regulatory factors, such as transcription factors, and DNA or RNA.

### Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labelling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR.

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte comprises a target sequence. In some embodiments, the target sequence may be endogenous to the sample, generated in the sample, added to the sample, or associated with an analyte in the sample. In some embodiments, the target sequence is a single-stranded target sequence (e.g., a sequence in a rolling circle amplification product). In some embodiments, the analytes comprise one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

### Labelling Agents

In some embodiments, provided herein are methods and compositions for analyzing endogenous analytes (e.g., RNA, ssDNA, and cell surface or intracellular proteins and/or metabolites) in a sample using one or more labelling agents. In some embodiments, an analyte labelling agent may include an agent that interacts with an analyte (e.g., an endogenous analyte in a sample). In some embodiments, the labelling agents can comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., a single-stranded probe sequence), that hybridizes to a reporter oligonucleotide of the labelling agent, in order to identify the analyte associated with the labelling agent. In some embodiments, the analyte labelling agent comprises an analyte binding moiety and a labelling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labelling agents.

In the methods and systems described herein, one or more labelling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labelling agents are the same. In some embodiments in which the plurality of analytes includes a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (i.e., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labelling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

### Products of Endogenous Analyte and/or Labelling Agent

In some embodiments, provided herein are methods and compositions for analyzing one or more products of an endogenous analyte and/or a labelling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some embodiments, a labelling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labelling agent that directly or indirectly binds to an analyte in the biological sample is analyzed.

### Hybridization

In some embodiments, a product of an endogenous analyte and/or a labelling agent is a hybridization product comprising the pairing of substantially complementary or complementary nucleic acid sequences within two different molecules, one of which is the endogenous analyte or the labelling agent (e.g., reporter oligonucleotide attached thereto). The other molecule can be another endogenous molecule or another labelling agent such as a probe. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labelling agent and each probe may comprise one or more barcode sequences. Exemplary barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary.

### Ligation

In some embodiments, a product of an endogenous analyte and/or a labelling agent is a ligation product. In some embodiments, the ligation product is formed between two or more endogenous analytes. In some embodiments, the ligation product is formed between an endogenous analyte and a labelling agent. In some embodiments, the ligation product is formed between two or more labelling agent. In some embodiments, the ligation product is an intramolecular ligation of an endogenous analyte. In some embodiments, the ligation product is an intramolecular ligation of a labelling agent, for example, the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labelling agent (e.g., the reporter oligonucleotide) or a product thereof.

In some embodiments, provided herein is a probe or probe set capable of DNA-templated ligation, such as from a cDNA molecule. See, e.g., U.S. Pat. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 20190055594, which is hereby incorporated by reference in its entirety. In some embodiments, provided herein is a multiplexed proximity ligation assay. See, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9°N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, i.e., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9, or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

### Primer Extension and Amplification

In some embodiments, a product is a primer extension product of an analyte, a labelling agent, a probe or probe set bound to the analyte (e.g., a padlock probe bound to genomic DNA, mRNA, or cDNA), or a probe or probe set bound to the labelling agent (e.g., a padlock probe bound to one or more reporter oligonucleotides from the same or different labelling agents).

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (i.e., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, a product of an endogenous analyte and/or a labelling agent is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling circle amplified to generate a cDNA nanoball (i.e., amplicon) containing multiple copies of the cDNA. Techniques for rolling circle amplification (RCA) are known in the art such as linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et aL, Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 11:1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, WO 2014/163886, WO 2017/079406, US 2016/0024555, US 2018/0251833 and US 2017/0219465. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template may comprise the target analyte, or a part thereof, where the target analyte is a nucleic acid, or it may be provided or generated as a proxy, or a marker, for the analyte. As noted above, many assays are known for the detection of numerous different analytes, which use a RCA-based detection system, e.g., where the signal is provided by generating a RCP from a circular RCA template which is provided or generated in the assay, and the RCP is detected to detect the analyte. The RCP may thus be regarded as a reporter which is detected to detect the target analyte. However, the RCA template may also be regarded as a reporter for the target analyte; the RCP is generated based on the RCA template and comprises complementary copies of the RCA template. The RCA template determines the signal, which is detected, and is thus indicative of the target analyte. As will be described in more detail below, the RCA template may be a probe, or a part or component of a probe, or may be generated from a probe, or it may be a component of a detection assay (i.e., a reagent in a detection assay), which is used as a reporter for the assay, or a part of a reporter, or signal-generation system. The RCA template used to generate the RCP may thus be a circular (e.g., circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some embodiments, a product herein includes a molecule or a complex generated in a series of reactions, e.g., hybridization, ligation, extension, replication, transcription/reverse transcription, and/or amplification (e.g., rolling circle amplification), in any suitable combination. For example, a product comprising a target sequence for a probe disclosed herein may be a hybridization complex formed of a cellular nucleic acid in a sample and an exogenously added nucleic acid probe. The exogenously added nucleic acid probe may comprise an overhang that does not hybridize to the cellular nucleic acid but hybridizes to another probe. The exogenously added nucleic acid probe may be optionally ligated to a cellular nucleic acid molecule or another exogenous nucleic acid molecule. In other examples, a product comprising a target sequence for a probe disclosed herein may be an RCP of a circularizable probe or probe set which hybridizes to a cellular nucleic acid molecule (e.g., genomic DNA or mRNA) or product thereof (e.g., a transcript such as cDNA, a DNA-templated ligation product of two probes, or an RNA-templated ligation product of two probes). In other examples, a product comprising a target sequence for a probe disclosed herein may a probe hybridizing to an RCP. The probe may comprise an overhang that does not hybridize to the RCP but hybridizes to another probe. The probe may be optionally ligated to a cellular nucleic acid molecule or another probe, e.g., an anchor probe that hybridize to the RCP.

### Target Sequences

A target sequence for a probe disclosed herein may be comprised in any analyte disclose herein, including an endogenous analyte (e.g., a viral or cellular nucleic acid), a labelling agent, or a product of an endogenous analyte and/or a labelling agent.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In any of the preceding embodiments, barcodes (e.g., primary and/or secondary barcode sequences) can be analyzed (e.g., detected or sequenced) using any suitable methods or techniques, including those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), in situ sequencing, hybridization-based in situ sequencing (HybISS), targeted in situ sequencing, fluorescent in situ sequencing (FISSEQ), sequencing by synthesis (SBS), sequencing by ligation (SBL), sequencing by hybridization (SBH), or spatially-resolved transcript amplicon readout mapping (STARmap). In any of the preceding embodiments, the methods provided herein can include analyzing the barcodes by sequential hybridization and detection with a plurality of labelled probes (e.g., detection oligos).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4^{N} complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and WO2019199579A1, which are hereby incorporated by reference in their entirety.

### In Situ Methods

The methods described herein may be useful for in situ-based methods in which specific reagents are added to a sample at one or more regions of interest. In situ methods include, e.g., in situ hybridization and in situ sequencing. In situ hybridization is a hybridization process in which labeled nucleic acids that are complementary to a specific nucleic acid (e.g., DNA or RNA) sequence in a biological sample hybridize to a portion or section of the sample (e.g., tissue) in which the nucleic acid is present. The in situ methods described herein may be used to detect and or quantify nucleic acids in a biological sample spatially by performing the method on the sample at one or more regions of interest. The in situ methods include using one or more fluid sources to flow in one or more reagents sequentially to contact the sample, e.g., at the region of interest, performing a hybridization and/or a chemical reaction with a labeled oligonucleotide, and detecting the label. Additional steps are described in more detail below.

The labeled nucleic acids, also referred to as probes, are generally short oligonucleotides in which at least a portion of the oligonucleotide is a reverse complement to a target nucleic acid of interest. The probes may include additional components in addition to the hybridization portion. For example, the probes may include additional sequences (e.g., barcode sequences), that are unique labels or identifiers to convey information about the nucleic acid being detected. The probes may further include a label attached thereto. The label may be, e.g., an optical label, a molecular label (e.g., an antigen), a radiolabel, or a field attractable label (e.g., electric or magnetic). In some embodiments the optical label is a fluorescent label, e.g., as used in fluorescence in situ hybridization (FISH). A fluorescent label can be detected by routine optical detection methods known in the art.

Optical detection may be performed by any detector capable of measuring light (e.g., the emitted, scattered, or attenuated light) from the label. Suitable detectors include, but are not limited to, a spectrometer, a light meter, a photometer, a photodiode, a photomultiplier tube, a CCD array, a CMOS sensor, or a photovoltaic device.

In situ methods may first include fixing and/or permeabilizing a biological sample (e.g., tissue). The biological sample may be fixed on the sample stage. The sample may be permeabilized by adding a fluid, such as a solvent (e.g., acetone and methanol) or a detergent (e.g., TRITON X-100, NP-40, TWEEN 20, saponin, digitonin, and Leucoperm), to the sample. Permeabilization may allow or enhance access of the probes for the intracellular space of the sample.

A probe may then be added to the sample, e.g., by flowing a fluid containing the probe through the inlet, to contact the biological sample (e.g., the sample medium containing the biological sample), e.g., at the region of interest. The probe hybridizes to the target, e.g., an mRNA. Any unbound probes may be washed away by flowing another fluid lacking the probe through the sample, e.g., via the inlet. The fluid containing the unbound probes may be removed from the sample, e.g., at region of interest, through the outlet of the device.

In some embodiments, a plurality of probes is used, e.g., for ease of detection and/or signal amplification. For example, a first probe may include a nucleic acid sequence that hybridizes to a target nucleic acid in the sample. A secondary probe that includes a label (e.g., optical label, e.g., fluorescent label) may then be added that hybridizes to the first probe. In some embodiments, a plurality of secondary or higher order (e.g., tertiary, quaternary) detection probes are added. Each probe may be provided by a separate fluid source. Each probe may be provided by a single fluid source that includes a plurality of distinct probes.

When a probe that includes a detection label is added, the unbound probes with detection labels can be washed away and the signal can be detected, e.g., via fluorescence microscopy.

In some embodiments, the signal or template target nucleic acid is amplified, e.g., by polymerase chain reaction (PCR) or rolling circle amplification (RCA). The target nucleic acid may be replicated, e.g., by using the probe as a primer to initiate DNA or RNA synthesis. In such an embodiment, one or more fluids are added (e.g., sequentially) to the sample to provide the reagents for nucleic acid synthesis. Suitable reagents include, but are not limited to, probes, primers, nucleotide triphosphates (NTPs, e.g., dNTPs), sequencing terminators, dyes, polymerases, ligases, transcriptases (e.g., reverse transcriptases), labels, and the like.

In some embodiments, following signal amplification, the sample may be embedded, e.g., in a hydrogel.

In some embodiments, the signal is increased by using a plurality of different probes that hybridize to the same nucleic acid, e.g., at a different sequential location. For example, an RNA transcript may contain a hybridization region for a plurality of (e.g., 2 ,3, 4, 6, 7, 8, 9, 10, 15, 20, 25, 30, 35, 40, 45, 50, or more) probes. Each probe or a secondary probe that hybridizes to the primary probe may contain a detectable label. This allows the plurality of labels all present on the same RNA to produce a detectable signal.

In some embodiments, the methods described herein includes in situ sequencing. One such process includes temporal multiplexing of barcoded FISH probes. This method, sometimes referred to as multiplexed error-robust fluorescence in situ hybridization (MERFISH) allows spatial transcriptome profiling of a large number of genes or an entire transcriptome (see, e.g., Moffitt et al, Meth. Enzymol. 572. 1-49, 2016, incorporated herein by reference). In this embodiment, a primary probe or set of primary probes (e.g., 24 primary probes) hybridize to a target nucleic acid (e.g., mRNA). Each probe may contain a barcode attached thereto. The barcodes may then be detected by performing a set of barcoding rounds in which the barcoded probe with a fluorescent label emits a signal. Each round of barcoding may be initiated by flowing the desired barcode label from a new fluid source, e.g., through the inlet of the device to the sample, e.g., at the region of interest. The labels may be detected using different excitation wavelengths (e.g., 640 nm, 561 nm, or 488 nm) during different barcoding rounds. By stitching together the spatiotemporal patterns of each fluorescent signal, the unique set of ordered barcode sequences that corresponds to a particular gene can be determined. Such a method may allow multiplex sequencing of a large number of (e.g., of 100, 1,000, 10,000, or more) nucleic acids, e.g., up to 90,000 transcripts per cell. This method also allows for efficient quantification of low-copy number nucleic acids. The steps may be performed on a first region of interest of the biological sample. Then, the steps may be repeated at one or more additional regions of interest in the sample, thus providing spatially informative transcriptome information.

In some embodiments, the in situ detection and/or in situ sequencing is performed in three dimensions. In this embodiment, the biological sample may be sequence by using a probe that includes a unique gene identifier. The probe may be or contain a nicked circle, which can be ligated, thereby allowing extension and amplification of the target sequence, e.g., by RCA. The amplification product can then be modified with a chemical moiety that polymerizes in the presence of a polymerization initiator. This forms an amplified product that is embedded within a polymerized matrix (e.g., a hydrogel), thereby creating a spatially fixed three-dimensional cDNA library of the biological sample.

In some embodiments, the in situ sequencing includes sequencing by ligation. In this embodiment, fluorescently labeled probes with two known bases followed by degenerate or universal bases hybridize to a temple. A ligase immobilizes the complex and the biological sample is imaged to detect the label on the probe. Following detection, the fluorophore is cleaved from the probe along with several bases, revealing a free 5′ phosphate. This process of hybridization, ligation, imaging, and cleavage can be repeating in multiple rounds, thereby allowing identification of, e.g., 2 out of every 5 bases. After a round of probe extension, all probes and anchors are removed and the cycle can begin again with an offset anchor, thus allowing sequencing of a new register of the target.

In another embodiment, sequencing by ligation includes labeled probes with a known base (e.g., A, C, T, or G) flanked on each side of the known base by degenerate or universal bases that hybridize to a template (e.g., three or four bases on each side). Each probe contains a different fluorescent label corresponding to each individual base. Each round of sequencing includes hybridizing a probe with a known base, ligation of the probe, detection, and optionally, cleavage of the fluorescent label.

Sequencing can be performed in a plus or minus direction, and rounds of sequencing can begin again with an offset anchor, thus allowing sequencing of a new register of the target.

Switch oligonucleotides (also referred to herein as “switch oligos” or “template switching oligonucleotides”) can be used for template switching. In some cases, template switching can be used to increase the length of a cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as template to further extend the cDNA. Template switching oligonucleotides may include a hybridization region and a template region. The hybridization region can comprise any sequence capable of hybridizing to the target. In some cases, as previously described, the hybridization region comprises a series of G bases to complement the overhanging C bases at the 3′ end of a cDNA molecule. The series of G bases may comprise 1 G base, 2 G bases, 3 G bases, 4 G bases, 5 G bases or more than 5 G bases. The template sequence can comprise any sequence to be incorporated into the cDNA. In some cases, the template region comprises at least 1 (e.g., at least 2, 3, 4, 5 or more) tag sequences and/or functional sequences. Switch oligos may comprise deoxyribonucleic acids; ribonucleic acids; modified nucleic acids including 2-Aminopurine, 2,6-Diaminopurine (2-Amino-dA), inverted dT, 5-Methyl dC, 2′-deoxyinosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′Fluoro bases (e.g., Fluoro C, Fluoro U, Fluoro A, and Fluoro G), or any combination.

In some cases, the length of a switch oligo may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197 , 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 nucleotides or longer.

In some cases, the length of a switch oligo may be at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197 , 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides or longer.

In some cases, the length of a switch oligo may be at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197 , 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides.

The macromolecular components (e.g., bioanalytes) of individual biological samples (e.g., cells) can be identified or detected with unique identifiers (e.g., barcodes) such that upon characterization of those macromolecular components, such that any given component (e.g., bioanalyte) may be traced to the biological sample (e.g., cell) from which it was obtained. The ability to attribute characteristics to individual biological samples or groups of biological samples is provided by the assignment of unique identifiers specifically to an individual biological sample or groups of biological samples. Unique identifiers, for example, in the form of nucleic acid barcodes, can be assigned or associated with individual biological samples (e.g., cells) or populations of biological samples (e.g., cells), or genes (e.g., mRNA transcripts, in order to tag or label the biological sample's macromolecular components (and as a result, its characteristics) with the unique identifiers. These unique identifiers can then be used to attribute the biological sample's components and characteristics to an individual biological sample or group of biological samples.

In some aspects, the unique identifiers are provided in the form of oligonucleotides that comprise nucleic acid barcode sequences that may be attached to or otherwise associated with the nucleic acid contents of individual biological sample, or to other components of the biological sample, and particularly to fragments of those nucleic acids.

The nucleic acid barcode sequences can include from 6 to about 20 or more nucleotides within the sequence of the oligonucleotides. In some cases, the length of a barcode sequence may be 6, 7, 8, 9, 10, 11, 12, 13, 14, 15, 16, 17, 18, 19, 20 nucleotides or longer. In some cases, the length of a barcode sequence may be at least 6, 7, 8, 9, 10, 11, 12, 13, 14, 15, 16, 17, 18, 19, 20 nucleotides or longer. In some cases, the length of a barcode sequence may be at most 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be 4, 5, 6, 7, 8, 9, 10, 11, 12, 13, 14, 15, 16 nucleotides or longer. In some cases, the barcode subsequence may be at least 4, 5, 6, 7, 8, 9, 10, 11, 12, 13, 14, 15, 16 nucleotides or longer. In some cases, the barcode subsequence may be at most 4, 5, 6, 7, 8, 9, 10, 11, 12, 13, 14, 15, 16 nucleotides or shorter.

Moieties (e.g., oligonucleotides) used in the methods described herein can also include other functional sequences useful in processing of nucleic acids from biological samples, e.g., contained in a droplet. These sequences include, for example, targeted or random/universal amplification primer sequences for amplifying the genomic DNA from the individual biological samples within the droplets while attaching the associated barcode sequences, sequencing primers or primer recognition sites, hybridization or probing sequences, e.g., for identification of presence of the sequences or for pulling down barcoded nucleic acids, or any of a number of other potential functional sequences.

The methods described herein may include providing molecular labels, e.g., via a fluid source. The molecular labels may include barcodes (e.g., nucleic acid barcodes). The molecular labels can be provided to the biological sample based on a number of different methods including, without limitation, microinjection, electroporation, liposome-based methods, nanoparticle-based methods, and lipophilic moiety-barcode conjugate methods. For instance, a lipophilic moiety conjugated to a nucleic acid barcode may be contacted with cells or particulate components of interest. The lipophilic moiety may insert into the plasma membrane of a cell thereby labeling the cell with the barcode. The devices and methods of the present invention may result in molecular labels being present on (i) the interior of a cell or particulate component and/or (ii) the exterior of a cell or particulate component (e.g., on or within the cell membrane). These and other suitable methods will be appreciated by those skilled in the art (see U.S. Pub. Nos. US20190177800, US20190323088, US20190338353, and US20200002763, each of which is incorporated herein by reference in its entirety).

In an example, a fluid is provided that includes large numbers of the above described barcoded oligonucleotides releasably attached to a label. In some cases, a fluid will provide a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more.

In some cases, it may be desirable to incorporate multiple different barcodes within a given sample. For example, in some cases, mixed, but known barcode sequences set may provide greater assurance of identification in the subsequent processing, for example, by providing a stronger address or attribution of the barcodes to a given droplet, as a duplicate or independent confirmation of the output from a given sample.

Oligonucleotides may be releasable from the labels (e.g., optical label, e.g., fluorescent label) upon the application of a particular stimulus. In some cases, the stimulus may be a photo-stimulus, e.g., through cleavage of a photo-labile linkage that releases the oligonucleotides. In other cases, a thermal stimulus may be used, where increase in temperature will result in cleavage of a linkage or other release of the oligonucleotides from the label. In still other cases, a chemical stimulus is used that cleaves a linkage of the oligonucleotides to the label, or otherwise results in release of the oligonucleotides from the label, e.g., beads.

### Definitions

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The term “about,” as used herein, refers to ±10% of a recited value.

The term “barcode,” as used herein, generally refers to a label, or identifier, that conveys or is capable of conveying information about an analyte. A barcode can be part of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads in real time.

The term “biological tissue sample” as used herein, refers to material from a subject, such as a biopsy, core biopsy, tissue section, needle aspirate, or fine needle aspirate or skin sample. The biological tissue sample may be derived from another sample.

The term “fluidically connected,” as used herein, refers to a direct connection between at least two device elements, e.g., a condenser channel, liquid trap, etc., that allows for fluid to move between such device elements without passing through an intervening element.

The term “genome,” as used herein, generally refers to genomic information from a subject, which may be, for example, at least a portion or an entirety of a subject's hereditary information. A genome can be encoded either in DNA or in RNA. A genome can comprise coding regions that code for proteins as well as non-coding regions. A genome can include the sequence of all chromosomes together in an organism. For example, the human genome has a total of 46 chromosomes. The sequence of all of these together may constitute a human genome.

The term “in fluid communication with,” as used herein, refers to a connection between at least two device elements, e.g., a condenser channel, liquid trap, etc., that allows for fluid to move between such device elements with or without passing through one or more intervening device elements.

The term “oil,” as used herein, generally refers to a liquid that is not miscible with water. An oil may have a density higher or lower than water and/or a viscosity higher or lower than water.

The term “region of interest,” as used herein, refers to a portion of a sample identified for fluid treatment.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA).

The term “subject,” as used herein, generally refers to an animal, such as a mammal (e.g., human) or avian (e.g., bird), or other organism, such as a plant. The subject can be a vertebrate, a mammal, a mouse, a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, or an individual that is in need of therapy or suspected of needing therapy. A subject can be a patient.

What is claimed is:

1. A device comprising a first layer and a second layer, wherein first layer comprises a window having a thickness of about 1 μm to about 1000 μm and wherein the first and second layers reversibly seal to form a flow path having an inlet and an outlet and bounded in part by the window.

2. The device of claim 1, wherein a thickness of the first layer bounding the window is greater than the thickness of the window.

3. The device of claim 1, wherein the thickness of the first layer bounding the window is from about 1 μm to about 1 mm.

4. The device of claim 1, wherein the thickness of the window is about 0.17 mm.

5. The device of claim 1, wherein the flow path comprises a void in a surface of the first or second layer.

6. The device of claim 1, wherein the first layer and the second layer are configured to seal via an adhesive, conformal contact, or capillary force.

7. The device of claim 1, wherein the first layer comprises a plurality of windows having a thickness of about 1 μm to about 1000 μm that are physically separated.

8. The device of claim 7, wherein each of the plurality of windows is separated by a region of the first layer having a greater thickness than each window.

9. The device of claim 7, wherein each of the plurality of windows is separated by a region of the first layer comprising a hydrophobic pattern.

10. The device of claim 1, wherein the flow path is bounded in part by a hydrophobic pattern on the first and/or second layer.

11. The device of claim 10, wherein the second layer comprises a hydrophobic surface texture or surface pattern opposite the window.

12. The device of claim 1, wherein the first and/or second layer comprises an elastomer.

13. The device of claim 1, wherein the second layer comprises the inlet and/or the outlet.

14. A system comprising the device of claim 1 and a clamp configured to apply a force on the device to maintain the seal.

15. A method for assembling a device comprising:
(a) providing the device of claim 1;
(b) applying a sample to the window; and
(c) reversibly contacting the first layer and the second layer and forming a fluid tight seal and the flow path through which fluid can flow.

16. The method of claim 15, wherein step (c) comprises applying a force on the device with the clamp.

17. A method for detection comprising:
(a) providing the device of claim 1;
(b) applying a sample to the window;
(c) flowing a fluid through the flow path, wherein the fluid comprises an oligonucleotide probe that hybridizes to a template nucleic acid in the sample; and
(d) detecting the oligonucleotide probe.

18. The method of claim 17, wherein the oligonucleotide probe comprises an optical label.

19. The method of claim 18, wherein the optical label is a fluorescent label.

20. The method of claim 17, further comprising flowing a fluid comprising a replicating enzyme and a plurality of nucleotide triphosphates (NTPs) or oligonucleotides to the sample.

21. The method of claim 20, wherein the replicating enzyme replicates the template nucleic acid with the NTPs.
